(19) United States
(12) Patent Application Publication
(10) Pub. No.: US 2003/0211497 A1
(43) Pub. Date: Nov. 13, 2003

(54) Diagnosis of schizohprenia by linkage of a polymorphic marker to a segment of chromosome 1q22 bordered by d1s2705 and d1s1679

(76) Inventors: Linda M Brzustowicz, Madison, NJ (US); Anne S Bassett, Toronto (CA)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/240,699

(22) PCT Filed: Apr. 5, 2001
(86) PCT No.: PCT/US01/10985

(51) Int. Cl. C12Q 1/68

(52) U.S. Cl. 435/6

(57) ABSTRACT

The invention maps a gene (SCZ) associated with schizophrenia to the q22 region of chromosome 1. The invention exploits this discovery to provide methods of diagnosing schizophrenia and schizophrenia susceptibility, methods of screening for the SCZ gene, and libraries of cloned segments including the SCZ gene.

This application claims priority under 35 U.S.C. §119(e) to U.S. Provisional Application 60/194,834 filed Apr. 5, 2000, the entire disclosure of which is incorporated by reference herein.

Pursuant to 35 U.S.C. §202(c) it is acknowledged that the U.S. Government has certain rights in the invention described herein, which was made in part with funds from the National Institutes of Mental Health, Grant Number: K08 MH01392.

### FIELD OF THE INVENTION

The present invention relates generally to the diagnosis and treatment of schizophrenia.

### BACKGROUND OF THE INVENTION

Schizophrenia is a serious neuropsychiatric illness estimated to affect 1.3% of the adult population in the United States (Report of the Surgeon General on Mental Health, 1999). The Diagnostic and Statistical Manual-IIIR (DSM-IIIR) criteria used to diagnose schizophrenia are provided hereinbelow in Table I. Age of onset is typically between age 15 and 25 for men, and between age 25 and 35 for women. The symptoms typically develop over weeks to months, with a prodromal period preceding the onset of acute psychotic symptoms. The disease is chronic, characterized by episodes of worsening symptoms with active psychosis, followed by periods of relative recovery marked by significant residual impairment. Current treatment is purely symptomatic, with no cure.

The lifetime risk for schizophrenia is 1.5 percent. Risk factors for schizophrenia include a history of schizophrenia in first-degree relatives, birth during the late winter months, and birth trauma. Patients with schizophrenia have substantial amounts of physical and psychological disability, as well as occupational difficulties, with disability equivalent to quadriplegia during periods of worsened symptoms (Report of the Surgeon General on Mental Health, 1999).

Schizoaffective disorder is a related syndrome characterized by the same disability and psychotic symptoms, but with the added feature of prevalent symptoms of mood disturbance. The DSM-IIIR diagnostic criteria (Table 2, set forth hereinbelow) describe this close relationship to schizophrenia. The lifetime prevalence of schizoaffective disorder is 0.5 to 0.8 percent.

A genetic component for schizophrenia has long been suggested. Family, twin and adoption studies have demonstrated that schizophrenia is predominantly genetic, with a high heritability (McGuffin et al., Br. J. Psychiatry 164:593, 1994). Segregation analyses have failed to clearly support a single model of inheritance, with the suggestion of at least several, possibly interacting, susceptibility loci (Risch, Hum. Genet. 46:222, 1990). Schizophrenia and schizoaffective disorder are often observed within the same family, suggesting that the two disorders may share a common genetic etiology. At present, no specific genetic or biochemical tests are available for the positive diagnosis of schizophrenia or schizoaffective disorder. Diagnosis and treatment is solely based on clinical evaluation. The clinical heterogeneity associated with schizophrenia and schizoaffective disorder has complicated the diagnosis and treatment of these disorders. Indeed, there is growing evidence that the episodes of severe psychotic symptoms may lead to irreversible decrements in long-term functioning. Current clinical trials have begun to treat individuals in the prodromal phase, with hopes of limiting the ultimate disability caused by these illnesses. Unfortunately, the diagnosis of schizophrenia or schizoaffective disorder cannot be accurately made during the prodromal phase. Additionally, the treatments carry a significant risk of serious side effects thus currently limiting this early intervention strategy to individuals known to be at extremely high risk for developing one of these disorders.

Identification of the inheritance pattern(s) and genetic bases for schizophrenia would greatly facilitate the diagnosis and treatment of this disorder. It is an object of the present invention to provide methods and kits which will aid the clinician in diagnosing this disorder.

### SUMMARY OF THE INVENTION

In accordance with the present invention, methods for diagnosing a patient having schizophrenia or schizoaffective disorder are provided. The term schizophrenia as used herein shall be interpreted to include both schizophrenia and the closely related schizoaffective disorder. In one embodiment of the invention, the presence or absence of an allele of a linked polymorphic marker in the DNA of the patient is determined. The polymorphic marker is present on chromosome 1q22 and is linked to a gene (SCZ) having a variant form associated with a phenotype of schizophrenia. The allele of the polymorphic marker detected in these methods is in phase with the variant form of the SCZ gene. Thus, the presence of the allele in the patient indicates susceptibility to schizophrenia. Closely linked polymorphic markers occur between D1S2705 and D1S1679. A preferred marker for use in the methods of the invention is B426K24T.

In an alternative embodiment, the methods disclosed comprise the additional step of determining the phase of the allele of the polymorphic marker detected in the patient with respect to the variant form of the SCZ gene. It is the variant form of the SCZ gene which leads to a schizophrenia phenotype. Phase can be established by determining the presence or absence of the allele in two relatives of the patient. Such relatives are preferably relatives of the first or second degree. The relatives should each be of known phenotype with respect to schizophrenia. At least one of the relatives should have schizophrenia, and the relatives should be informative for the marker. The phenotype of relatives can be determined from the criteria of the Diagnostic and Statistical Manual IIIR shown in Tables I and II.

In a further embodiment of the invention, susceptibility to schizophrenia in a patient is determined by analyzing a relative of the patient for a phenotype of schizophrenia. These methods are particularly useful when the patient is presently asymptomatic or exhibiting marginal symptoms.

In yet another embodiment of the invention, kits are provided for the diagnosis of schizophrenia. Such a kit comprises an oligonucleotide which hybridizes to a DNA segment within chromosome 1q22, the DNA segment being linked to the SCZ gene. Preferably, the oligonucleotide hybridizes to a DNA segment between D1S2705 and D1S1679. In one aspect, the kit comprises paired first and second oligonucleotides for amplification of a target segment DNA. The first and second oligonucleotides serve to prime amplification of a target DNA segment between D1S2705 and D1S1679. In another aspect, the kits comprise paired first and second oligonucleotides respectively hybridizing to first and second allelic variants of the DNA segment of the invention. Such kits are useful in methods which include, but are not limited to ASO analysis or allele-specific PCR.

The invention also provides libraries enriched for clones from the region of chromosome 1q22 containing the SCZ gene. The libraries consist essentially of a plurality of vectors each encoding a segment of DNA between D1S2705 and D1S1679.

In a further embodiment of the invention, methods for screening and isolation of the SCZ gene are provided. In this aspect, cDNA or genomic DNA sequences from individuals with schizophrenia and known to carry a defect in the SCZ gene by virtue of genetic linkage to chromosome 1q22 are screened for alterations in DNA sequence. These differences are then compared to the DNA sequence in normal individuals. Methods for screening patient DNA for these alterations include without limitation, direct DNA sequencing, single strand conformation polymorphism analysis (SSCP), heteroduplex analysis (HA), chemical cleavage of mismatched sequences (CCMS), denaturing gradient gel electrophoresis (DGGE), temperature gradient gel electrophoresis (TGGE), denaturing high performance liquid chromatography (dHPLC), ribonuclease cleavage, carbodiimide modification, and microarray analysis. The SCZ encoding nucleic acid isolated using any of the foregoing methods is also encompassed within the present invention.

The following definitions are provided to facilitate an understanding of the present invention:

The term “corresponds to” is used herein to mean that a polynucleotide sequence is homologous to all or a portion of a reference polynucleotide sequence, or that a polypeptide sequence is identical to a reference polypeptide sequence. In contradistinction, the term “complementary to” is used herein to mean that the complementary sequences is homologous to all or a portion of a reference polynucleotide sequence. For illustration, the nucleotide sequence “TATAC” corresponds to a reference sequence “TATAC” and is complementary to a reference sequence “GTATA”. Hybridization probes may be DNA or RNA, or any synthetic nucleotide structure capable of binding in a base-specific manner to a complementary strand of nucleic acid. For example, probes include peptide nucleic acids, as described in Nielsen et al., Science 254:1497-1500 (1991).

“Linkage” describes the tendency of genes, alleles, loci or genetic markers to be inherited together as a result of their location on the same chromosome, and is measured by percent recombination (also called recombination fraction, or θ) between the two genes, alleles, loci or genetic markers.

“Centimorgan” is a unit of genetic distance signifying linkage between two genetic markers, alleles, genes or loci, corresponding to a probability of recombination between the two markers or loci of 1% for any meiotic event.

“Linkage disequilibrium” or “allelic association” means the-preferential association of a particular allele, locus, gene or genetic marker with a specific allele, locus, gene or genetic marker at a nearby chromosomal location more frequently than expected by chance for any particular allele frequency in the population.

An “oligonucleotide” can be DNA or RNA, and single- or double-stranded. Oligonucleotides can be naturally occurring or synthetic, but are typically prepared by synthetic means.

The term “primer” refers to an oligonucleotide capable of acting as a point of initiation of DNA synthesis under conditions in which synthesis of a primer extension product complementary to a nucleic acid strand is induced, i.e., in the presence of four different nucleoside triphosphates and an agent for polymerization (i.e., DNA polymerase or reverse transcriptase) in an appropriate buffer and at a suitable temperature. A primer is preferably a single-stranded oligonucleotide. The appropriate length of a primer depends on the intended use of the primer but typically ranges from 15 to 30 nucleotides. Short primer molecules generally require cooler temperatures to form sufficiently stable hybrid complexes with the template. A primer need not reflect the exact sequence of the template but must be sufficiently complementary to hybridize with a template. The term “primer” may refer to more than one primer, particularly in the case where there is some ambiguity in the information regarding one or both ends of the target region to be amplified. For instance, if a region shows significant levels of polymorphism or mutation in a population, mixtures of primers can be prepared that will amplify alternate sequences. A primer can be labeled, if desired, by incorporating a label detectable by spectroscopic, photochemical, biochemical, immunochemical, or chemical means. For example, useful labels include ³²P, fluorescent dyes, electron-dense reagents, enzymes (as commonly used in an ELISA), biotin, or haptens and proteins for which antisera or monoclonal antibodies are available. A label can also be used to “capture” the primer, so as to facilitate the immobilization of either the primer or a primer extension product, such as amplified DNA, on a solid support.

“Chromosome 1 set” means the two copies of chromosome 1 found in somatic cells or the one copy in germ line cells of a patient or family member. The two copies of chromosome 1 may be the same or different at any particular allele, including alleles at or near the schizophrenia locus. The chromosome 1 set may include portions of chromosome 1 collected in chromosome 1 libraries, such as plasmid, yeast, or phage libraries, as described in Sambrook et al., Molecular Cloning, 2nd Edition, and in Mandel et al., Science 258:103-108 (1992).

“Penetrance” is the percentage of individuals with a defective gene who show some symptoms of a trait resulting from that defect. Expressivity refers to the degree of expression of the trait (e.g., mild, moderate or severe).

“Polymorphism” refers to the occurrence of two or more genetically determined alternative sequences or alleles in a population. A polymorphic marker is the locus at which divergence occurs. Preferred markers have at least two alleles, each occurring at frequency of greater than 1%. A polymorphic locus may be as small as one base pair. Polymorphic markers suitable for use in the invention include restriction fragment length polymorphisms, variable number of tandem repeats (VNTR's), hypervariable regions, minisatellites, dinucleotide repeats, trinucleotide repeats, tetranucleotide repeats, and other microsatellite sequences.

“Restriction fragment length polymorphism” (RFLP) means a variation in DNA sequence that alters the length of a restriction fragment as described in Botstein et al.; Am. J. Hum. Genet. 32:314-331 (1980). The restriction fragment length polymorphism may create or delete a restriction site, thus changing the length of the restriction fragment. For example, the DNA sequence GAATTC are the six bases, together with its complementary strand CTTAAG which comprises the recognition and cleavage site of the restriction enzyme EcoRI. Replacement of any of the six nucleotides on either strand of DNA to a different nucleotide destroys. the EcoRI site. This RFLP can be detected by, for example, amplification of a target sequence including the polymorphism, digestion of the amplified sequence with EcoRI, and size fractionation of the reaction products on an agarose or acrylamide gel. If the only EcoRI restriction enzyme site within the amplified sequence is the polymorphic site, the target sequences comprising the restriction site will show two fragments of predetermined size, based on the length of the amplified sequence. Target sequences without the restriction enzyme site will only show one fragment, of the length of the amplified sequence. Similarly, the RFLP can be detected by probing an EcoRI digest of Southern blotted DNA with a probe from a nearby region such that the presence or absence of the appropriately sized EcoRT fragment may be observed. RFLP's may be caused by point mutations which create or destroy a restriction enzyme site, VNTR's, dinucleotide repeats, deletions, duplications, or any other sequence-based variation that creates or deletes a restriction enzyme site, or alters the size of a restriction fragment.

“Variable number of tandem repeats” (VNTR's) are short sequences of nucleic acids arranged in a head to ) tail fashion in a tandem array, and found in each individual, as described in Wyman et al., Proc. Nat. Acad. Sci. 77:6754-6758 (1980). Generally, the VNTR sequences are comprised of a core sequence of at least 16 base pairs, with a variable number of repeats of that sequence. Additionally, there may be variation within the core sequence, Jefferys et al., Nature 314:67-72 (1985). These sequences are highly individual, and perhaps unique to-each individual. Thus, VNTR's may generate restriction fragment length polymorphisms, and ) may additionally serve as size-based amplification product differentiation markers.

“Microsatellite sequences” comprise segments of at least about 10 base pairs of DNA consisting of a variable number of tandem repeats of short (1-6 base pairs) sequences of DNA(Clemens et al., Am. J. Hum. Genet. 49:951-960 1991). Microsatellite sequences are generally spread throughout the chromosomal DNA of an individual. The number of repeats in any particular tandem array varies greatly from individual to individual, and thus, microsatellite sequences may serve to generate restriction fragment length polymorphisms, and may additionally serve as size-based amplification product differentiation markers.

A “marker”[is referred to as fully “informative” for a particular individual if the configuration of alleles observed in the family allow for the unambiguous determination of parental origin of the alleles of a child. For example, if the mother has a “1” and “2” allele, while the father has a “3”, and “4” allele, then it is possible to unambiguously assign the parental origin of alleles in each of the four possible combinations in the children (1-3, 1-4, 2-3, 2-4). A marker is partially informative when unambiguous determination of parental origin is possible for only certain children. For example, if both parents have a “1” and “2” allele, then the parental origins of the alleles may be unambiguously determined for children with the genotypes 1-1 and 2-2, but not for the children with the genotype 1-2. If one parent is homozygous for a marker, the marker will be only partially informative, and the inheritance from that parent cannot be traced. If the marker is homozygous in both parents, the marker is fully uninformative for the transmission from them to their children, even though their children may be heterozygous and thus informative for the transmission of that marker to the next generation.

### BRIEF DESCRIPTION OF THE DRAWINGS

FIG. 1 is a graph showing multipoint linkage results with markers APOA2, D1S2675, and D1S1679.

FIGS. 2A-2E depict haplotype analysis of SCZ segregation with polymorphic markers in five families containing key recombination events which localize the SCZ gene. An upward arrow indicates proximal localization of the SCZ gene and a downward arrow indicates distal localization of the SCZ gene.

### DETAILED DESCRIPTION OF THE INVENTION

I. Methods of Diagnosis

The present invention provides methods of identifying patients having a variant allele of a gene associated with the schizophrenia phenotype. The gene (SCZ) is located in human chromosome 1 in the region conventionally designated q22 by reference to cytological markers and DNA. See Weissenbach et al., Nature 359:794 (1992); Gyapay et al., Nature Genetics 7:246 (1994); Murray, CHLC Report (1994). Specifically, the gene is within a segment of about 5 cM between polymorphic markers D1S2705 and D1S1679. An allele of the gene present in persons not suffering from schizophrenia is arbitrarily designated as wildtype. A variant allele of the gene is associated with a phenotype of schizophrenia. Such genetic variants include, without limitation, nucleotide additions, deletions or substitutions relative to the wildtype allele. These genetic alterations are associated with a phenotype of schizophrenia, as defined by the Diagnostic and Statistical Manual (DSM)-IIIR criteria (see Example 1) in at least some individuals bearing the variant allele. The phenotype may result from a nucleotide change in the gene (addition, deletion or substitution) affecting expression of the gene by altering the kinetics of expression or the nature of the resulting expression product. For example, some changes reduce transcription or translation of an expression product. Other changes result in a polypeptide having altered properties (cf. the sickle cell mutation). Still other changes introduce a premature stop codon thereby resulting in truncated expression product.

A substantial proportion of patients having two variant copies of SCZ experience symptoms of schizophrenia or, alternatively, are at high risk for developing these symptoms later in life. The genetic tests of the present invention provide a highly accurate assay for diagnosing schizophrenia and schizophrenia susceptibility. Physicians having the correct diagnosis in hand can then ensure that patients receive prophylactic or therapeutic treatment appropriate to the genetic and biochemical bases of the disease.

The methods may also be used to advantage for in utero screening of fetuses for the presence of a variant SCZ allele. Identification of such variations offers the possibility of gene therapy. For couples known to be at risk of giving rise to affected progeny, diagnosis can be combined with in vitro reproduction procedures to identify an embryo having wildtype SCZ alleles before implantation. Screening children shortly after birth is also of value in identifying those having the variant gene. Early detection allows administration of appropriate treatment.

A. Mode of Inheritance

Example 4 presents evidence that a schizophrenia susceptibility gene can be inherited in an autosomal recessive fashion. The autosomal recessive mode of inheritance is unexpected given that the families used in this study demonstrated patterns of disease segregation that would be more consistent with autosomal dominant inheritance. However, a common recessive allele can produce patterns of inheritance in families that resemble autosomal dominant inheritance.

This recognition is of immediate benefit in diagnosing an asymptomatic patient with a relative suffering from schizophrenia in a family, some of whose members have schizophrenia associated with the SCZ gene. It is apparent that the patient is also at risk of having acquired the variant allele(s) associated with the disease, and subsequently developing symptoms of the disease. For example, if the patient has a sibling suffering from schizophrenia, the odds of the patient having acquired the same variant alleles are 25%. The odds of the patient actually developing the disease are probably less than 25% because of incomplete penetrance of the disease. For example, at a penetrance of 50%, the odds of the patient developing the disease would be 12.5%.

B. Diagnosis from Linked Polymorphic Markers

The invention further provides methods of diagnosing susceptibility to schizophrenia by detection of polymorphic markers linked to the SCZ gene on human chromosome 1. Markers are linked if they occur within 50 cM from each other or the SCZ gene. Preferably, markers occur within 15 cM and more preferably within 5 or 1 cM of the gene. The closer the polymorphic marker is to SCZ locus, the less likely there is to be physical recombination between the two loci at meiosis. The polymorphic marker is usually outside the SCZ gene, but also may occur within the gene. All human chromosomes are subdivided into regions by cytological and polymorphic markers. Example 4 shows that preferred markers include those mapped between D1S2705 and D1S1679, including APOA2, FcGR2A, FcER1G, B426K24T and D1S2675. Publications providing a detailed description of these polymorphic markers except B426K24T from the q22 region of chromosome 1 are provided in Table 3 and incorporated by reference in their entirety herein. The B426K24T marker is described in Example 3. D1S1679 shows the strongest linkage of markers tested to date. Thus, this marker and other markers within about 5 cM of it are preferred for use in the methods of the present invention. Most preferred are markers which occur within the SCZ gene itself. The claimed methods are utilized to determine which alleles of a linked polymorphic marker are present in the patient being diagnosed. For example, if the polymorphic marker is an RFLP, the alleles differ in the size of a restriction fragment. The determination is typically made by PCR amplification of a segment spanning the polymorphism and gel analysis of the amplification product. If one of the alleles present in the patient is known to be in phase with a variant SCZ locus (i.e., present on the same chromosome), it is concluded with a high probability that the patient has a variant SCZ gene and is susceptible to developing schizophrenia. The closer linked the polymorphic marker to SCZ, the higher the probability that the patient has received the variant SCZ gene. See Sutherland & Mulley, Clinical Genetics 37:2-11 (1990). Preferably, the methods analyze the presence of alleles of two polymorphic markers spaced on either side of the SCZ gene and both in phase with the gene. Absent a rare double recombination event, the presence of both alleles signals the presence of the variant SCZ gene.

The method described above requires knowledge that a particular allele of a marker is in phase with the variant form of the SCZ gene. This information is acquired from analyzing the phenotype and polymorphic content of relatives of the patient in a family, some of whose members exhibit schizophrenia. The linkage and/or phase determinations are usually performed before analysis of DNA from the patient. Linkage can be established by any of the methods discussed in Example 4.

Determinations of linkage and/or phase are usually performed before analysis of DNA from the patient. A phase determination requires at least two relatives of the patient who are of known phenotype for schizophrenia, at least one of the relatives having the disease and being informative for the marker. In practice, a relative having the disease is screened at several polymorphic markers to identify at least one marker in which the relative is heterozygous. The phase of this marker is then set by determining which alleles of the marker are present in a second relative of known phenotype. Strategies for setting phase in different families are described by Lazarou, Clinical Genetics 43:150-156 (1993). For example, consider two siblings, X (with disease) having alleles 1 and 2 of a marker linked to the disease, and Y (without disease) having alleles 3 and 4. It can be concluded that in this family, the 1 and 2 alleles are in phase with the variant SCZ gene. As a further example, consider X (with disease) having alleles 1 and 2 and Y (with disease) having alleles 1 and 5. It is deduced that the 1, 2 and 5 alleles are in phase with the variant gene. Within a family, the allele of a closely linked marker that is in phase with the variant gene is usually the same in each affected family member because there is a low probability of recombination between the two loci. The more closely related the relatives to the patient, the more likely phase is to be conserved between the relatives and the patient. Thus, it is preferred that one of the relatives used in setting phase is a parent or sibling of the patient. Once phase has been determined for a family, multiple members of the family can be diagnosed without repeating the analysis. In general, the phase relationship between an allele of a polymorphic marker and a variant allele of the SCZ gene is different in each family. However, certain alleles may be in linkage disequilibrium with the SCZ gene. For such markers, the same allele is likely to be in phase with the variant allele of the SCZ gene in any family. Thus, once such an allele is identified it is not necessary to set phase in every family to be tested.

C. Direct Assays for SCZ Gene

Having localized the SCZ gene as described infra, variations can be detected by more direct methods. These methods represent a special case of the methods described above in which the polymorphic marker being detected is a variation arising within the SCZ gene.

1. Detection of Uncharacterized Variations

Hitherto uncharacterized variations in the SCZ gene are identified and localized to specific nucleotides by comparison of nucleic acids from an individual with schizophrenia with an unaffected individual, preferably a relative of the affected individual. Comparison with a relative is preferred because the possibility of other polymorphic differences between the patient and person being compared, not related to the schizophrenia phenotype, is lower. Various screening methods are suitable for this comparison including, but not limited to, direct DNA sequencing, single strand conformation polymorphism analysis (SSCP), heteroduplex analysis (HA), chemical cleavage of mismatched sequences (CCMS), denaturing gradient gel electrophoresis (DGGE), temperature gradient gel electrophoresis (TGGE), denaturing high performance liquid chromatography (dHPLC), ribonuclease cleavage, carbodiimide modification, and microarray analysis. See Cotton, Mutation Res. 285:125-144 (1993). Comparison can be initiated at either cDNA or genomic level. Initial comparison is often easier at the cDNA level because of its shorter size. Corresponding genomic changes are then identified by amplifying and sequencing a segment from the genomic exon including the site of change in the cDNA. In some instances, there is a simple relationship between genomic and cDNA changes. That is, a single base change in a coding region of genomic DNA gives rise to a corresponding changed codon in the cDNA. In other instances, the relationship between genomic and cDNA changes is more complex. Thus, for example, a single base change in genomic DNA creating an aberrant splice site can give rise to deletion of a substantial segment of cDNA.

2. Detection of Characterized Changes

The preceding methods serve to identify particular genetic changes responsible for schizophrenia. In any particular family, it is likely that all affected members have the same change. Individuals from different families may or may not have the same change. However, typically, many individuals have one of a relatively small number of changes. By analogy, in cystic fibrosis, about seventy percent of individuals have the same mutation in the CFTR gene. Once a change has been identified within a family, and/or as occurring within a population of affected individuals at a significant frequency, individuals can be tested for that change by various methods. These methods include allele-specific oligonucleotide hybridization, allele-specific amplification, ligation, primer extension and artificial introduction of extension sites (see Cotton, supra). For example, the allele-specific detection method uses one oligonucleotide exhibiting a perfect match to a target segment of the SCZ gene having the change and a paired probe exhibiting a perfect match to the corresponding wildtype segment. If the individual is homozygous wildtype, only the wildtype probe binds. If the individual is a heterozygous variant, both probes bind. If the individual is a homozygous variant, only the variant probe binds. Paired probes for several variations can be immobilized as an array and the presence of several variations can thereby be analyzed simultaneously. Of course, the methods noted above, for analyzing uncharacterized variations can also be used for detecting characterized variations.

II. Identification of the SCZ Gene

In accordance with the present invention methods of screening for the SCZ gene are also provided. The position of the SCZ gene can be localized by haplotype analysis as described in Example 4. See also Current Protocols in Human Genetics (eds. Dracopli et al., Wiley, 1994), Unit 1.3 (incorporated by reference in its entirety herein). In this analysis, the phenotype with respect to schizophrenia is determined for successive generations of family members. Family members are then tested to determine which alleles are present for polymorphic markers mapping close to the SCZ gene (i.e., between D1S2705 and D1S1679). The alleles present are assigned to one of the two copies of chromosome 1 present in the individual whereby the number of recombination events between successive generations of the family is minimized. This analysis reveals which of the two copies of chromosome 1 an individual has received from each parent, and where, if at all, a recombination event has occurred in this chromosome in the region of interest. By identifying a site of recombination between members of successive generations in a family, and knowing whether the members share or differ in the schizophrenia phenotype, the location of the SCZ gene relative to the site of recombination (i.e., on one side or the other) is revealed. The SCZ gene is described as “proximal” to the site of recombination (or a marker bordering the site of recombination), if the gene occurs between the site of recombination (or the marker) and the centromere. The SCZ gene is described as “distal” to the site of recombination (or the marker), if the gene occurs between the site of recombination (or the marker) and the telomere. The site of recombination can vary between different generations and between different families. Thus, the possible positions in which the SCZ gene can occur consistent with its proximal or distal nature with respect to each point of recombination identified is progressively confined as more families are tested.

Having localized the SCZ gene to a small segment within the q22 region of chromosome 1, the region can then be mapped for restriction sites by pulsed field gel electrophoresis. A library is then prepared and enriched for clones mapping to this region. Chromosomal segments are preferably cloned into BAC vectors. Such vectors offer a capacity of up to 200 kb per vector. Thus, relatively few clones are required to cover the entire segment to which the SCZ gene has been localized. As a starting material for preparing such a library, a library of the whole human genome is already available. Clones mapping to the region of interest can be isolated by, e.g., chromosome walking. Briefly, a first marker bordering the segment of interest is used as a probe to identify a first clone containing sequence complementary to the probe. A second probe is then designed based on the sequence of the first clone at the end nearest the SCZ gene. The second probe is then used to isolate a second clone, which is in turn used to design a third probe. The process continues until a clone is isolated which hybridizes to a second marker, known to be on the distal side of the SCZ gene from the first marker. See Wainwright, Med. J. Australia 159:170-174 (1993); DOE, Primer On Molecular Genetics (Washington D.C., June 1992); Collins, Nature Genetics 1:3-6 (1992) (each of which is incorporated by reference in its entirety herein). BACs known to map to the region between D1S2705 and D1S1679 include, without limitation, those listed in Table 5 under Example 5.

Preferably, a small library of clones completely spanning the region of interest is obtained, which is substantially free (at least 75% free) of clones having segments mapping elsewhere in chromosome 1. The region of interest is bordered by D1S2705 and D1S1679, and is about 2 Mb is length. Segments spanning the 0.75 Mb between B426K24T and D1S2675 are of particular interest. Typically, a library spanning 1 Mb of human DNA contains approximately 25 genes. The clones are sequenced to search for open-reading frames and analyzed for transcription by Northern blotting, in situ hybridization, zoo-blotting (probing with xenogeneic DNA to identify conserved sequences), exon trapping (Davies, supra) and/or HTF-island mapping (CCGG sites associated with the 5′ end of many genes). Alternatively, putative coding sequences can be identified from lengths of DNA sequence by gene prediction software and then verified by identification within an appropriate cDNA library. Having identified an open reading frame that appears to be expressed, this region of DNA is compared between affected and unaffected members of a family to identify the presence of variations that correlate with the disease phenotype.

III. Expression Systems

Identification of the SCZ gene facilitates the production of the gene product. The cDNA fragment or any other nucleic acid encoding the SCZ gene can be used to make an expression construct for the SCZ gene. The expression construct typically comprises one or more nucleic acid sequences encoding the SCZ gene operably linked to a native or other promoter. Usually, the promoter is a eukaryotic promoter for expression in a mammalian cell. The transcription regulation sequences typically include a heterologous enhancer or promoter which is recognized by the host. The selection of an appropriate promoter, for example trp, lac, phage promoters, glycolytic enzyme promoters and tRNA promoters, depends on the host selected. Commercially available expression vectors can be used. Vectors can include host-recognized replication systems, amplifiable genes, selectable markers, host sequences useful for insertion into the host genome, and the like.

The means of introducing the expression construct into a host cell varies depending upon the particular vector and targeted host cell. Suitable means include fusion, conjugation, transfection, transduction, electroporation or injection, as described in Sambrook, supra. A wide variety of host cells can be employed for expression of the SCZ gene, both prokaryotic and eukaryotic. Suitable host cells include bacteria such as E. coli, yeast, filamentous fungi, insect cells, mammalian cells, typically immortalized, e.g., mouse, CHO, human and monkey cell lines and derivatives thereof. Preferred host cells are able to process the SCZ gene product to produce an appropriate mature polypeptide. Processing includes glycosylation, ubiquitination, disulfide bond formation, general post-translational modification, and the like.

The SCZ protein may be isolated by conventional means of protein biochemistry and purification to obtain a substantially pure product, i.e., 80, 95 or 99% free of cell component contaminants, as described in Jacoby, Methods in Enzymology Volume 104, Academic Press, New York (1984); Scopes, Protein Purification, Principles and Practice, 2nd Edition, Springer-Verlag, New York (1987); and Deutscher (ed), Guide to Protein Purification, Methods in Enzymology, Vol. 182 (1990). If the protein is secreted, it can be isolated from the supernatant in which the host cell is grown. If not secreted, the protein can be isolated from a lysate of the host cells.

The invention further provides transgenic nonhuman animals capable of expressing an exogenous SCZ gene and/or having one or both alleles of an endogenous SCZ gene inactivated. Expression of an exogenous SCZ gene is usually achieved by operably linking the gene to a promoter and optionally an enhancer, and microinjecting the construct into a zygote. See Hogan et al., “Manipulating the Mouse Embryo, A Laboratory Manual,” Cold Spring Harbor Laboratory. Inactivation of endogenous SCZ genes can be achieved by forming a transgene in which a cloned SCZ gene is inactivated by insertion of a positive selection marker. See Capecchi, Science 244:1288-1292 (1989). The transgene is then introduced into an embryonic stem cell, where it undergoes homologous recombination with an endogenous SCZ gene. Mice and other rodents are preferred animals. Such animals provide useful in vivo drug screening systems.

In addition to substantially full-length polypeptides expressed by the SCZ gene, the present invention includes biologically active fragments of the polypeptides, or analogs thereof, including organic molecules which simulate the interactions of the peptides. Biologically active fragments include any portion of the full-length polypeptide which confers a biological function on the SCZ gene product, including ligand binding, substrate for other molecules, dimer association, and the like. Ligand binding includes binding by nucleic acids, proteins or polypeptides, small biologically active molecules, or large cellular structures.

Polyclonal and/or monoclonal antibodies to the SCZ gene product are also provided. Antibodies can be made by injecting mice or other animals with the SCZ gene product or synthetic peptide fragments thereof. monoclonal antibodies are screened by methods known in the art, as are described, for example, in Harlow & Lane, Antibodies, A Laboratory Manual, Cold Spring Harbor Press, New York (1988), and Goding, Monoclonal antibodies, Principles and Practice (2d ed.) Academic Press, New York (1986). Monoclonal antibodies are tested for specific immunoreactivity with an epitope of the SCZ gene product. These antibodies are useful in diagnostic assays for detection of the SCZ gene product or a variant form thereof, or as an active ingredient in a pharmaceutical composition.

IV. Methods of Treatment

There are a number of drugs presently in use for treating schizophrenia. However, no clear distinctions have been drawn between schizophrenia patients in prescribing decisions. The present discovery that at least some subtypes of schizophrenia are associated with common genetic and presumably, biochemical features allows drug screening programs to be conducted in a group of patients-having homogeneous disposition with respect to the SCZ gene. Such a group is identified by the diagnostic methods discussed above.

The provision of DNA encoding the SCZ gene is also useful in developing new drugs and methods of treatment for schizophrenia. For example, variations in the SCZ gene, including regulatory sequences, can be corrected by gene therapy. See Rosenberg, J. Clin. Oncol. 10:180-199 (1992). Gene therapy is preferably performed in utero rather than after birth, because of the undifferentiated nature of cells in a developing fetus. Exogenously supplied corrective genes integrate into the genomes of undifferentiated cells, and are subsequently distributed and expressed in entire tissues by the proliferation and differentiation of the ancestor cell.

The provision of the SCZ gene product also allows screening for a receptor or soluble molecules that interact with the same and design of agents that agonize or antagonize this interaction. Such agents include monoclonal antibodies against the SCZ gene product, fragments of the SCZ gene product that compete with the full-length protein for binding, and synthetic peptides or analogs thereof selected from random combinatorial libraries. See, e.g., Ladner et al., U.S. Pat. No. 5,223,409 (1993) (incorporated by reference in its entirety herein). Therapeutic agents also includes transcription factors, and the like, which stimulate expression of the SCZ gene.

V. Diagnostic Kits

The present invention also includes kits for the practice of the methods of the invention. The kits comprise a vial, tube, or any other container which contains one or more oligonucleotides, which hybridizes to a DNA segment within chromosome 1q22, which DNA segment is linked to the SCZ gene. Preferably, the oligonucleotide hybridizes to a segment of chromosome 1 between markers D1S2705 and D1S1679. Some kits contain two such oligonucleotides, which serve as primers to amplify a segment of chromosome DNA. The segment selected for amplification can be a polymorphic marker linked to the SCZ gene or a region from the SCZ gene that includes a site at which a variation is known to occur. Some kits contain a pair of oligonucleotides for detecting precharacterized variations. For example, some kits contain oligonucleotides suitable for allele-specific oligonucleotide hybridization, or allele-specific amplification hybridization. The kits of the invention may also contain components of the amplification system, including PCR reaction materials such as buffers and a thermostable polymerase. In other embodiments, the kit of the present invention can be used in conjunction with commercially available amplification kits, such as may be obtained from GIBCO BRL (Gaithersburg, Md.) Stratagene (La Jolla, Calif.), Invitrogen (San Diego, Calif.), Schleicher & Schuell (Keene, N.H.), Boehringer Mannheim (Indianapolis, Ind.). The kits may optionally include positive or negative control reactions or markers, molecular weight size markers for gel electrophoresis, and the like. The kits usually include labelling or instructions indicating the suitability of the kits for diagnosing schizophrenia and indicating how the oligonucleotides are to be used for that purpose. The term “label” is used generically to encompass any written or recorded material that is attached to, or otherwise accompanies the diagnostic at any time during its manufacture, transport, sale or use.

Modes of Practicing the Invention

1. Linkage Analysis

Determining linkage between a polymorphic marker and a locus associated with a particular phenotype is performed by mapping polymorphic markers and observing whether they co-segregate with the schizophrenia phenotype on a chromosome in an informative meiosis. See, e.g., Kerem et al., Science 245:1073-1080 (1989); Monaco et al., Nature 316:842 (1985); Yamoka et al., Neurology 40:222-226 (1990), and as reviewed in Rossiter et al., FASEB Journal 5:21-27 (1991). A single pedigree rarely contains enough informative meioses to provide definitive linkage, because families are often small and markers may be not sufficiently informative. For example, a marker may not be polymorphic in a particular family.

Linkage may be established by an affected sib-pairs analysis as described in Terwilliger & Ott, Handbook of Human Genetic Linkage (Johns Hopkins, Md., 1994), Ch. 26. This approach requires no assumptions to be made concerning penetrance or variant frequency, but only takes into account the data of a relatively small proportion (i.e., the SIB pairs) of all the family members whose phenotype and polymorphic markers have been determined. Specifically, the affected SIB pairs analysis scores each pair of affected SIBS as sharing (concordant) or not sharing (discordant) the same allelic variant of each polymorphic marker. For each marker, a probability is then calculated that the observed ratio of concordant to discordant SIB pairs would arise without linkage of the marker.

As described in Thompson & Thompson, Genetics in Medicine, 5th ed, 1991, W. B. Saunders Company, Philadelphia, in linkage analysis, one calculates a series of likelihood ratios (relative odds) at various possible values of θ, ranging from θ=0.0 (no recombination) to θ=0.50 (random assortment). Thus, the likelihood ratio at a given value of θ is (likelihood of data if αloci are linked at θ/(likelihood of data if loci are unlinked). Evidence in support of linkage is usually expressed as the log₁₀ of this ratio and called a “lod score” for “logarithm of the odds.” For example, a lod score of 5 indicates 100,000:1 odds that the linkage being observed did not occur by chance.

The use of logarithms allows data collected from different families to be combined by simple addition. Computer programs are available for the calculation of lod scores for differing values of θ. Available programs include LIPED, and MLINK (Lathrop, Proc. Nat. Acad. Sci. 81:3443-3446 (1984).

For any particular lod score, a recombination fraction may be determined from mathematical tables. See Smith et al., Mathematical tables for research workers in human genetics (Churchill, London, 1961) and Smith, Ann. Hum. Genet. 32:127-150 (1968). The value of θ at which the lod score is the highest is considered to be the best estimate of the recombination fraction, the “maximum likelihood estimate”.

Positive lod score values suggest that the two loci are linked, whereas negative values suggest that linkage is less likely (at that value of θ) than the possibility that the two loci are unlinked. By convention, a combined lod score of +3 or greater (equivalent to greater than 1000:1 odds in favor of linkage) is considered definitive evidence that two loci are linked. Similarly, by convention, a negative lod score of −2 or less is taken as definitive evidence against linkage of the two loci being compared. If there are sufficient negative linkage data, a locus can be excluded from an entire chromosome, or a portion thereof, a process referred to as exclusion mapping. The search is then focused on the remaining non-excluded chromosomal locations. For a general discussion of lod scores and linkage analysis, see, e.g., T. Strachan, Chapter 4, “Mapping the human genome” in The Human Genome, 1992 BIOS Scientific Publishers Ltd. Oxford.

The data can also be subjected to haplotype analysis. This analysis assigns allelic markers between the chromosomes of an individual such that the number of recombinational events needed to account for segregation between generations is minimized. Linkage may also be established by determining the relative likelihood of obtaining observed segregation data for any two markers when the two markers are located at a recombination fraction θ, versus the situation in which the two markers are not linked, and thus segregating independently.

2. Isolation and Amplification of DNA

Samples of patient, proband or family member genomic DNA is isolated from any convenient source including saliva, buccal cells, hair roots, blood, cord blood, amniotic fluid, interstitial fluid, peritoneal fluid, chorionic villus, and any other suitable cell or tissue sample with intact interphase nuclei or metaphase cells. The cells can be obtained from solid tissue as from a fresh or preserved organ or from a tissue sample or biopsy. The sample can contain compounds which are not naturally intermixed with the biological material such as preservatives, anticoagulants, buffers, fixatives, nutrients, antibiotics, or the like.

Methods for isolation of genomic DNA from these various sources are described in, for example, Kirby, DNA Fingerprinting, An Introduction, W. H. Freeman & Co. New York (1992). Genomic DNA can also be isolated from cultured primary or secondary cell cultures or from transformed cell lines derived from any of the aforementioned tissue samples.

Samples of patient, proband or family member RNA can also be used. RNA can be isolated from tissues expressing the SCZ gene as described in Sambrook et al., supra. RNA can be total cellular RNA, mRNA, poly A+ RNA, or any combination thereof. For best results, the RNA is purified, but can also be unpurified cytoplasmic RNA. RNA can be reverse transcribed to form DNA which is then used as the amplification template, such that the PCR indirectly amplifies a specific population of RNA transcripts. See, e.g., Sambrook, supra, Kawasaki et al., Chapter 8 in PCR Technology, (1992) supra, and Berg et al., Hum. Genet. 85:655-658 (1990).

3. PCR Amplification

The most common means for amplification is polymerase chain reaction (PCR), as described in U.S. Pat. Nos. 4,683,195, 4,683,202, 4,965,188 each of which is hereby incorporated by reference. If PCR is used to amplify the target regions in blood cells, heparinized whole blood should be drawn in a sealed vacuum tube kept separated from other samples and handled with clean gloves. For best results, blood should be processed immediately after collection; if this is impossible, it should be kept in a sealed container at 4° C. until use. Cells in other physiological fluids may also be assayed. When using any of these fluids, the cells in the fluid should be separated from the fluid component by centrifugation.

Tissues should be roughly minced using a sterile, disposable scalpel and a sterile needle (or two scalpels) in. a 5 mm Petri dish. Procedures for removing paraffin from tissue sections are described in a variety of specialized handbooks well known to those skilled in the art.

To amplify a target nucleic acid sequence in a sample by PCR, the sequence must be accessible to the components of the amplification system. One method of isolating target DNA is crude extraction which is useful for relatively large samples. Briefly, mononuclear cells from samples of blood, amniocytes from amniotic fluid, cultured chorionic villus cells, or the like are isolated by layering on sterile Ficoll-Hypaque gradient by standard procedures. Interphase cells are collected and washed three times in sterile phosphate buffered saline before DNA extraction. If testing DNA from peripheral blood lymphocytes, an osmotic shock (treatment of the pellet for 10 sec with distilled water) is suggested, followed by two additional washings if residual red blood cells are visible following the initial washes. This will prevent the inhibitory effect of the heme group carried by hemoglobin on the PCR reaction. If PCR testing is not performed immediately after sample collection, aliquots of 10⁶ cells can be pelleted in sterile Eppendorf tubes and the dry pellet frozen at −20° C. until use.

The cells are resuspended (10⁶ nucleated cells per 100 μl) in a buffer of 50 mM Tris-HCl (pH 8.3), 50 mM KCl 1.5 mM MgCl₂, 0.5% Tween 20, 0.5% NP40 supplemented with 100 μg/ml of proteinase K. After incubating at 56° C. for 2 hr, the cells are heated to 95° C. for 10 min to inactivate the proteinase K and immediately moved to wet ice (snap-cool). If gross aggregates are present, another cycle of digestion in the same buffer should be undertaken. Ten μl of this extract is used for amplification.

When extracting DNA from tissues, e.g., chorionic villus cells or confluent cultured cells, the amount of the above mentioned buffer with proteinase K may vary according to the size of the tissue sample. The extract is incubated for 4-10 hrs at 50°-60° C. and then at 95° C. for 10 minutes to inactivate the proteinase. During longer incubations, fresh proteinase K should be added after about 4 hr at the original concentration.

When the sample contains a small number of cells, extraction may be accomplished by methods as described in Higuchi, “Simple and Rapid Preparation of Samples for PCR”, in PCR Technology, Ehrlich, H. A. (ed.), Stockton Press, New York, which is incorporated herein by reference. PCR can be employed to amplify target regions chromosome 1 in very small numbers of cells (1000-5000) derived from individual colonies from bone marrow and peripheral blood cultures. The cells in the sample are suspended in 20 μl of PCR lysis buffer (10 mM Tris-HCl (pH 8.3), 50 mM KCl, 2.5 mM MgCl₂, 0.1 mg/ml gelatin, 0.45% NP40, 0.45% Tween 20) and frozen until use. When PCR is to be performed, 0.6 μl of proteinase K (2 mg/ml) is added to the cells in the PCR lysis buffer. The sample is then heated to about 60° C. and incubated for 1 hr. Digestion is stopped through inactivation of the proteinase K by heating the samples to 95° C. for 10 min and then cooling on ice.

A relatively easy procedure for extracting DNA for PCR is a salting out procedure adapted from the method described by Miller et al., Nucleic Acids Res. 16:1215 (1988), which is incorporated herein by reference. Mononuclear cells are separated on a Ficoll-Hypaque gradient. The cells are resuspended in 3 ml of lysis buffer (10 mM Tris-HCl, 400 nM NaCl, 2 mM Na₂ EDTA, pH 8.2). Fifty μl of a 20 mg/ml solution of proteinase K and 150 μl of a 20% SDS solution are added to the cells and then incubated at 37° C. overnight. Rocking the tubes during incubation will improve the digestion of the sample. If the proteinase K digestion is incomplete after overnight incubation (fragments are still visible), an additional 50 μl of the 20 mg/ml proteinase K solution is mixed in the solution and incubated for another night at 37° C. on a gently rocking or rotating platform. Following adequate digestion, one ml of a 6M NaCl solution is added to the sample and vigorously mixed. The resulting solution is centrifuged for 15 minutes at 3000 rpm. The pellet contains the precipitated cellular proteins, while the supernatant contains the DNA. The supernatant is removed to a 15 ml tube that contains 4 ml of isopropanol. The contents of the tube are mixed gently until the water and the alcohol phases have mixed and a white DNA precipitate has formed. The DNA precipitate is removed and dipped in a solution of 70% ethanol and gently mixed. The DNA precipitate is removed from the ethanol and air-dried. The precipitate is placed in distilled water and dissolved.

Kits for the extraction of high-molecular weight DNA for PCR include a Genomic Isolation Kit A.S.A.P. (Boehringer Mannheim, Indianapolis, Ind.), Genomic DNA Isolation System (GIBCO BRL, Gaithersburg, Md.), Elu-Quik DNA Purification Kit (Schleicher & Schuell, Keene, N.H.), DNA Extraction Kit (Stratagene, La Jolla, Calif.), TurboGen Isolation Kit (Invitrogen, San Diego, Calif.), and the like. Use of these kits according to the manufacturer's instructions is generally acceptable for purification of DNA prior to practicing the methods of the present invention.

The concentration and purity of the extracted DNA can be determined by spectrophotometric analysis of the absorbance of a diluted aliquot at 260 nm and 280 nm. After extraction of the DNA, PCR amplification may proceed. The first step of each cycle of the PCR involves the separation of the nucleic acid duplex formed by the primer extension. Once the strands are separated, the next step in PCR involves hybridizing the separated strands with primers that flank the target sequence. The primers are then extended to form complementary copies of the target strands. For successful PCR amplification, the primers are designed so that the position at which each primer hybridizes along a duplex sequence is such that an extension product synthesized from one primer, when separated from the template (complement), serves as a template for the extension of the other primer. The cycle of denaturation, hybridization, and extension is repeated as many times as necessary to obtain the desired amount of amplified nucleic acid.

In a particularly useful embodiment of PCR amplification, strand separation is achieved by heating the reaction to a sufficiently high temperature for an sufficient time to cause the denaturation of the duplex but not to cause an irreversible denaturation of the polymerase (see U.S. Pat. No. 4,965,188, incorporated herein by reference). Typical heat denaturation involves temperatures ranging from about 80° C. to 105° C. for times ranging from seconds to minutes. Strand separation, however, can be accomplished by any suitable denaturing method including physical, chemical, or enzymatic means. Strand separation may be induced by a helicase, for example, or an enzyme capable of exhibiting helicase activity. For example, the enzyme RecA has helicase activity in the presence of ATP. The reaction conditions suitable for strand separation by helicases are known in the art (see Kuhn Hoffman-Berling, 1978, CSH-Quantitative Biology, 43:63-67; and Radding, 1982, Ann. Rev. Genetics 16:405-436, each of which is incorporated herein by reference).

Template-dependent extension of primers in PCR is catalyzed by a polymerizing agent in the presence of adequate amounts of four deoxyribonucleotide triphosphates (typically dATP, dGTP, dCTP, and dTTP) in a reaction medium comprised of the appropriate salts, metal cations, and pH buffering systems. Suitable polymerizing agents are enzymes known to catalyze template-dependent DNA synthesis.

In some cases, the target regions may encode at least a portion of a protein expressed by the cell. In this instance, mRNA may be used for amplification of the target region. Alternatively, PCR can be used to generate a cDNA library from RNA for further amplification, the initial template for primer extension is RNA. Polymerizing agents suitable for synthesizing a complementary, copy-DNA (cDNA) sequence from the RNA template are reverse transcriptase (RT), such as avian myeloblastosis virus RT, Moloney murine leukemia virus RT, or Thermus thermophilus (Tth) DNA polymerase, a thermostable DNA polymerase with reverse transcriptase activity marketed by Perkin Elmer Cetus, Inc. Typically, the genomic RNA template is heat degraded during the first denaturation step after the initial reverse transcription step leaving only DNA template. Suitable polymerases for use with a DNA template include, for example, E. coli DNA polymerase I or its Klenow fragment, T4 DNA polymerase, Tth polymerase, and Taq polymerase, a heat-stable DNA polymerase isolated from Thermus aquaticus and commercially available from Perkin Elmer Cetus, Inc. The latter enzyme is widely used in the amplification and sequencing of nucleic acids. The reaction conditions for using Taq polymerase are known in the art and are described in Gelfand, 1989, PCR Technology, supra.

4. Allele Specific PCR

Allele-specific PCR differentiates between chromosome 1 target regions differing in the presence or absence of a variation or polymorphism. PCR amplification primers are chosen which bind only to certain alleles of the target sequence. Thus, for example, amplification products are generated from those chromosome 1 sets which contain the primer binding sequence, and no amplification products are generated in chromosome 1 sets without the primer binding sequence. This method is described by Gibbs, Nucleic Acid Res. 17:12427-2448 (1989).

5. Allele Specific Oligonucleotide Screening Methods

Further diagnostic screening methods employ the allele-specific oligonucleotide (ASO) screening methods, as described by Saiki et al., Nature 324:163-166 (1986). Oligonucleotides with one or more base pair mismatches are generated for any particular allele. ASO screening methods detect mismatches between variant target genomic or PCR amplified DNA and non-mutant oligonucleotides, showing decreased binding of the oligonucleotide relative to a mutant oligonucleotide. Oligonucleotide probes can be designed that under low stringency will bind to both polymorphic forms of the allele, but which at higher stringency, bind to the allele to which they correspond. Alternatively, stringency conditions can be devised in which an essentially binary response is obtained, i.e., an ASO corresponding to a variant form of the SCZ gene will hybridize to that allele, and not to the wildtype allele.

6. Ligase Mediated Allele Detection Method

Target regions of a patients can be compared with target regions in unaffected and affected family members by ligase-mediated allele detection. See Landegren et al., Science 241:1077-1080 (1988). Ligase may also be used to detect point mutations in the ligation amplification reaction described in Wu et al., Genomics 4:560-569 (1989). The ligation amplification reaction (LAR) utilizes amplification of specific DNA sequence using sequential rounds of template dependent ligation as described in Wu, supra, and Barany, Proc. Nat. Acad. Sci. 88:189-193 (1990).

7. Denaturing Gradient Gel Electrophoresis

Amplification products generated using the polymerase chain reaction can be analyzed by the use of denaturing gradient gel electrophoresis. Different alleles can be identified based on the different sequence-dependent melting properties and electrophoretic migration of DNA in solution. DNA molecules melt in segments, termed melting domains, under conditions of increased temperature or denaturation. Each melting domain melts cooperatively at a distinct, base-specific melting temperature (Tm). Melting domains are at least 20 base pairs in length, and may be up to several hundred base pairs in length.

Differentiation between alleles based on sequence specific melting domain differences can be assessed using polyacrylamide gel electrophoresis, as described in Chapter 7 of Erlich, ed., PCR Technology, Principles and Applications for DNA Amplification, W. H. Freeman and Co, New York (1992), the contents of which are hereby incorporated by reference.

Generally, a target region to be analyzed by denaturing gradient gel electrophoresis is amplified using PCR primers flanking the target region. The amplified PCR product is applied to a polyacrylamide gel with a linear denaturing gradient as described in Myers et al., Meth. Enzymol. 155:501-527 (1986), and Myers et al., in Genomic Analysis, A Practical Approach, K. Davies Ed. IRL Press Limited, Oxford, pp. 95-139 (1988), the contents of which are hereby incorporated by reference. The electrophoresis system is maintained at a temperature slightly below the Tm of the melting domains of the target sequences.

In an alternative method of denaturing gradient gel electrophoresis, the target sequences may be initially attached to a stretch of GC nucleotides, termed a GC clamp, as described in Chapter 7 of Erlich, supra. Preferably, at least 80% of the nucleotides in the GC clamp are either guanine or cytosine. Preferably, the. GC clamp is at least 30 bases long. This method is particularly suited to target sequences with high Tm's.

Generally, the target region is amplified by the polymerase chain reaction as described above. One of the oligonucleotide PCR primers carries at its 5′ end, the GC clamp region, at least 30 bases of the GC rich sequence, which is incorporated into the 5′ end of the target region during amplification. The resulting amplified target region is run on an electrophoresis gel under denaturing gradient conditions as described above. DNA fragments differing by a single base change will migrate through the gel to different positions, which may be visualized by ethidium bromide staining.

8. Temperature Gradient Gel Electrophoresis

Temperature gradient gel electrophoresis (TGGE) is based on the same underlying principles as denaturing gradient gel electrophoresis, except the denaturing gradient is produced by differences in temperature instead of differences in the concentration of a chemical denaturant. Standard TGGE utilizes an electrophoresis apparatus with a temperature gradient running along the electrophoresis path. As samples migrate through a gel with a uniform concentration of a chemical denaturant, they encounter increasing temperatures. An alternative method of TGGE, temporal temperature gradient gel electrophoresis (TTGE or tTGGE) uses a steadily increasing temperature of the entire electrophoresis gel to achieve the same result. As the samples migrate through the gel the temperature of the entire gel increases, leading the samples to encounter increasing temperature as they migrate through the gel. Preparation of samples, including PCR amplification with incorporation of a GC clamp, and visualization of products are the same as for denaturing gradient gel electrophoresis.

9. Single-Strand Conformation Polymorphism Analysis

Target sequences or alleles at the SCZ locus can be differentiated using single-strand conformation polymorphism analysis, which identifies base differences by alteration in electrophoretic migration of single stranded PCR products, as described in Orita et al., Proc. Nat. Acad. Sci. 86:2766-2770 (1989). Amplified PCR products can be generated as described above, and heated or otherwise denatured, to form single stranded amplification products. Single-stranded nucleic acids may refold or form secondary structures which are partially dependent on the base sequence. Thus, electrophoretic mobility of single-stranded amplification products can detect base-sequence difference between alleles or target sequences.

10. Chemical or Enzymatic Cleavage of Mismatches

Differences between target sequences can also be detected by differential chemical cleavage of mismatched base pairs, as described in Grompe et al., Am. J. Hum. Genet. 48:212-222 (1991). In another method, differences between target sequences can be detected by enzymatic cleavage of mismatched base pairs, as described in Nelson et al., Nature Genetics 4:11-18 (1993). Briefly, genetic material from a patient and an affected family member may be used to generate mismatch free heterohybrid DNA duplexes. As used herein, “heterohybrid” means a DNA duplex strand comprising one strand of DNA from one person, usually the patient, and a second DNA strand from another person, usually an affected or unaffected family member. Positive selection for heterohybrids free of mismatches allows determination of small insertions, deletions or other polymorphisms that may be associated with schizophrenia.

11. Non-PCR Based DNA Diagnostics

The identification of a DNA sequence linked to SCZ can made without an amplification step, based on polymorphisms including restriction fragment length polymorphisms in a patient and a family member. Hybridization probes are generally oligonucleotides which bind through complementary base pairing to all or part of a target nucleic acid. Probes typically bind target sequences lacking complete complementarity with the probe sequence depending on the stringency of the hybridization conditions. The probes are preferably labeled directly or indirectly, such that by assaying for the presence or absence of the probe, one can detect the presence or absence of the target sequence. Direct labeling methods include radioisotope labeling, such as with ³²P or ³⁵S. Indirect labeling methods include fluorescent tags, biotin complexes which may be bound to avidin or streptavidin, or peptide or protein tags. Visual detection methods include photoluminescents, Texas red, rhodamine and its derivatives, red leuco dye and 3, 3′, 5, 5′-tetramethylbenzidine (TMB), fluorescein, and its derivatives, dansyl, umbelliferone and the like or with horse radish peroxidase, alkaline phosphatase and the like.

Hybridization probes include any nucleotide sequence capable of hybridizing to the 1q22 region of chromosome 1, and thus defining a genetic marker linked to SCZ, including a restriction fragment length polymorphism, a hypervariable region, repetitive element, or a variable number tandem repeat. Hybridization probes can be any gene or a suitable analog. Further suitable hybridization probes include exon fragments or portions of cDNAs or genes known to map to the q22 region of chromosome 1. Other suitable probes include portions of introns or intron/exon spanning regions from genomic fragments of chromosome 1, or portions of spacer DNA, i.e., DNA between genes that is not intronic.

Preferred tandem repeat hybridization probes for use according to the present invention are those that recognize a small number of fragments at a specific locus at high stringency hybridization conditions, or that recognize a larger number of fragments at that locus when the stringency conditions are lowered.

The following examples are provided to illustrate various embodiments of the present invention. They are not intended to limit the invention in any way.

### EXAMPLE I

Diagnosis of Schizophrenia in Study Families

Persons enrolling in the present study were referred by regional psychiatrists who first identify potential new families with two or more affected members. A key family historian was then interviewed to obtain an outline of the pedigree and an indication of the probable willingness of family members to participate in the study. Pedigrees were included if at least two living adult members had DSM-III-R schizophrenia or chronic schizoaffective disorder and did not meet the exclusion criteria of predominant bipolar affective disorder or known organic or physical disturbances causing major psychiatric illnesses. The DSM-IIIR criteria for schizophrenia are summarized in Table 1. The DSM-IIIR criteria for schizoaffective disorder are summarized in Table 2.

TABLE I
Diagnostic Criteria for Schizophrenia

| | |
|---|---|
| A. | Presence of characteristic psychotic symptoms in the |

active phase: either 1), 2) or 30 for at least one week
(unless the symptoms are successfully treated):
1) two of the following:

a) delusions
b) prominent hallucinations (throughout the day for
several days or several times a week for several
weeks, each hallucinatory experience not being
limited to a few brief moments)
c) incoherence or marked loosening of associations
d) catatonic behavior
e) flat or grossly inappropriate affect

2) bizarre delusions involving a phenomenon that the
person's culture would regard as totally implausible
(e.g., thought broadcasting, being controlled by a dead
person)
3) prominent hallucinations (as defined in 1)b) above) of
a voice with content having no apparent relations to
depression or elation, or a voice keeping up a running
commentary on the person's behavior or thoughts, or two
or more voices conversing with each other.

| | |
|---|---|
| B. | During the course of the disturbance, functioning in |

such areas as work, social relations, and self-care is
markedly below the highest level achieved before onset
of the disturbance (or when the onset is in childhood or
adolescence, failure to achieve expected level of social
development

| | |
|---|---|
| C. | Schizoaffective disorder and mood disorder with |

psychotic features have been ruled out, i.e., if a major
depressive or manic syndrome has ever been present
during an active phase of the disturbance, the total
durations of all episodes of a mood syndrome has been
brief relative to the total duration of the active and
residual phases of the disturbance.

| | |
|---|---|
| D. | Continuous signs of the disturbance for at least six |

months. The six-month period must include an active
phase (of at least one week or less if symptoms have
been successfully treated) during which there were
psychotic symptoms characteristic of schizophrenia
(symptoms in A), with or without a prodromal or residual
phase as defined below.

| | |
|---|---|
| E. | Prodromal phase: A clear deterioration in |

functioning before the active phase of the disturbance
that is not due to a disturbance in mood or to a
psychoactive substance use disorder and that involves at
least two of the symptoms listed below.

| | |
|---|---|
| F. | Residual phase: Following the active phase of the |

disturbance, persistence of at least two of the symptoms
noted below, these not being due to a disturbance in
mood or to a psychoactive substance use disorder.
Prodromal or residual symptoms:

| | |
|---|---|
| 1) | marked social isolation or withdrawal |
| 2) | marked impairment in role functioning as wage- |
| | earner, student or homemaker |
| 3) | markedly peculiar behavior (e.g., collecting |
| | garbage, talking to oneself in public, hoarding |
| | food) |
| 4) | marked impairment in personal hygiene and grooming |
| 5) | blunted or inappropriate affect |
| 6) | digressive, vague, overelaborate, or circumstantial |
| | speech, or poverty of speech, or poverty of content |
| | of speech |
| 7) | odd beliefs or magical thinking, influencing |
| | behavior and inconsistent with cultural norms |
| | (e.g., superstitiousness, belief in clairvoyance, |
| | telepathy, “sixth sense” “others can feel my |
| | feelings” overvalued ideas, ideas of reference) |
| 8) | unusual perceptual experiences (e.g., recurrent |
| | illusions, sensing the presence of a force or |
| | person not actually present) |
| 9) | marked lack of initiative, interests or energy |

Examples: Six months of a prodromal symptoms with one
week of symptoms from A; no prodromal symptoms with six
months of symptoms from A, no prodromal symptoms with
one week of symptoms from A and six months of residual
symptoms
It cannot be established that an organic factor
initiated and maintained the disturbance.
If there is a history of autistic disorder, the
additional diagnosis of schizophrenia is made only if
prominent delusions or hallucinations are also present.

TABLE II
Diagnostic Criteria for Schizoaffective Disorder

| | |
|---|---|
| A. | A disturbance during which, at some time, there is |

either a major depressive or a manic syndrome concurrent
with symptoms that meet a criterion of schizophrenia.

| | |
|---|---|
| B. | During an episode of the disturbance, there have |

been delusions or hallucinations for at least two weeks,
but not prominent mood symptoms.

| | |
|---|---|
| C. | Schizophrenia has been ruled out (i.e., the duration |

of all episodes of a mood syndrome has not been brief
relative to the total duration of the psychotic
disturbance).

| | |
|---|---|
| D. | It cannot be established that an organic factor |

initiated and maintained the disturbance.

Individual family members meeting inclusion criteria (18 years old or older, English speaking, willing to participate in diagnostic interviews and venipuncture) were then scheduled for interviews with a project psychiatrist. The Structured Clinical Interviews for DSM-III-R (Diagnostic & Statistical Manual of Mental Disorders, Third Edition, Revised) SCID-I and SCID-II (Spitzer et al., Structural Clinical Interview for DSM-III-R-Patient Edition (SCID-P, Version 1.0) (American Psychiatric Press, Washington, 1990), were the chief diagnostic instruments for this study, providing DSM-III-R Axis I (major psychiatric disorder) and Axis II (personality disorder, including schizophrenia spectrum conditions) diagnoses, respectively. They were chosen as comprehensive structured interview schedules, with the advantage of being based on a clinical interview. Sufficient data were collected to use other diagnostic classification schemes, including Research Diagnostic Criteria, DSM-IV, and ICD-10. To capture the full phenotypic spectrum and to delineate premorbid and comorbid conditions, all sections of the SCID-I were used. The SCID-II was rearranged and shortened to highlight paranoid, schizoid and schizotypal features, and the Structured Interview for Schizotypy (SIS; Kendler et al., Schizophr. Bull. 15:559-571, 1989) was used as a supplementary guide to better assess schizophrenia spectrum conditions. Personal history and observational data collected on mental status examination, essential to diagnose schizotypal features, are of high quality because psychiatrists experienced with schizophrenia and related disorders were assessing subjects.

Extensive data were obtained from each subject on long term functioning, symptoms, personal history, and medical history, useful for differential diagnosis and for determining schizophrenia spectrum conditions. Complete mental status examination (MSE) narratives provided qualitative observations on behavior, speech, affect and abstract thinking. A Mini-Mental Status examination (MMSE; Folstein et al., 1975) provided additional objective information on cognitive functioning. The Positive and Negative Syndrome Scale (PANSS; Kay et al., Schizophr. Bull. 13:261-275, 1987) quantitatively assessed key symptom groupings. An Abnormal Involuntary Movement Screen (AIMS) was also included. Aside from these direct assessments, data was collected by the family history method on as many relatives as each subject knew, using the Family History-Research Diagnostic Criteria (FH-RDC; Andreasen et al., Arch. Gen. Psychiatry, Arch. Gen. Psychiatry, 34: 1228-1235, 1977). This important collateral information extends data on personality characteristics, behavior and functioning.

The psychiatrists performing the direct interviews in this study are experienced with all of the assessment instruments and have high inter-rater reliability. For a genetic study, it is imperative to have experienced clinicians performing the interviews, to maximize accuracy in diagnostic assessment. Interviewers were blind to marker genotype but not to familial relationships, given the nature of the interviews. The interviews took place either in the subject's home or at a nearby clinic and were audiotaped if the subject consented (90%). The project psychiatrist, usually with the research assistant, conducted the diagnostic interviews, performed mental status examinations, and collected collateral information. Following the interview, the psychiatrist scored the PANSS, wrote the MSE and made the field diagnosis. Subjects were assessed when they were not in illness episodes and symptoms were therefore most likely to be stable. Subjects were re-interviewed and further medical records obtained if major changes, e.g., first hospitalization for psychosis, occured. New diagnoses, taking the complete longitudinal history into account, were then made.

The research assistant obtained medical records on all subjects with a history of hospitalization, made copies and removed names and all information pertaining to familial relationship. Genealogical records, where available, were searched for verification of family history information and extension of the pedigree. Hospital records, where available, were searched for evidence of mental illness in earlier generations, and abstracted as described above. Folders containing interview data, medical records, narrative summaries, and collateral information were compiled for each subject. Audiotapes were available for diagnostic clarification.

The interviewing psychiatrist, in discussion with the other project psychiatrist, then made a consensus field diagnoses based on the total contents of the diagnostic folder, attached a level of certainty with respect to meeting criteria, and recorded differential diagnoses. Folders containing all available clinical information, purged of references to name, familial relationship and diagnoses assigned were then reviewed by an independent psychiatrist, who was blind to the pedigree structure. Interview information and all collateral data were used to determine the Best Estimate Clinical Evaluation and Diagnosis (BECED). If the BECED diagnosis agreed with the consensus field diagnosis, this became the research diagnosis used for the linkage analysis. Following suggested guidelines (Weeks et al., Schizophr. Bull. 16:673-686, 1990; Maziade et al., Am. J. Psychiatry, 149: 1674-1686, 1992) if the BECED diagnosis disagreed with the field diagnosis, a diagnostic panel of three psychiatrists independently determines a BECED, following collection of more follow-up or collateral data.

The analysis identified 22 families with schizophrenia or schizoaffective disorder in at least two individuals. All available first-degree relatives (parents, siblings and children) of affected individuals age 18 or older were interviewed, with diagnoses assigned as above. Overall, 304 subjects were evaluated, with 79 meeting diagnostic criteria for schizophrenia or schizoaffective disorder.

For 288 subjects, genomic DNA was prepared and analyzed as described in Examples 2 and 3 with 384 markers with an average heterozygosity of 0.76 which span the genome at an average density of one marker per 9 cM. In addition, all subjects were genotyped with the chromosome 1 markers D1S1653, D1S398, D1S2635, D1S2771, D1S2705, APOA2, D1S2768, D1S2844, and D1S1677 (Table 3). These markers span approximately 12 cM on chromosome 1. In addition, a subset of subjects was genotyped with the markers FcGR2A and FcER1G (Table III).

TABLE III
Human Chromosome 1 Multiallelic Markers

| Locus | Gene | Reference |
|---|---|---|
| D1S1653 | DNA segment | GDB Human Genome Data Base |
| D1S398 | DNA segment | GDB Human Genome Data Base |
| D1S2635 | DNA segment | Nature 380: 152-154, 1996 |
| D1S2771 | DNA segment | Nature 380: 152-154, 1996 |
| D1S2705 | DNA segment | Nature 380: 152-154, 1996 |
| APOA2 | apolipoprotein A-II | GDB Human Genome Data Base |
| FcER1G | Fc receptor, IgE, | GDB Human Genome Data Base |
| | high affinity I, | |
| | gamma polypeptide | |
| FcGR2A | Fc receptor, IgG, | GDB Human Genome Data Base |
| | low affinity IIa | |
| | polypeptide | |
| D1S2675 | DNA segment | Nature 380: 152-154, 1996 |
| D1S1679 | DNA segment | GDB Human Genome Data Base |
| D1S2768 | DNA segment | Nature 380: 152-154, 1996 |
| D1S2844 | DNA segment | Nature 380: 152-154, 1996 |
| D1S1677 | DNA segment | GDB Human Genome Data Base |

### EXAMPLE 2

Preparation of Genomic DNA

Approximately 30 ml of blood was collected from each family member into tubes containing K₂-EDTA or other anticoagulant. DNA was extracted from these samples using the GenePure system (Gentra Systems). Red blood cells were lysed by addition of 3 volumes of RBC Lysis Solution (Gentra Systems), and the remaining white blood cells were pelleted by centrifugation. The white blood cells were lysed with 1 volume of Cell Lysis Solution (Gentra Systems), and treated with RNase A at 37° C. for 15 minutes. After cooling, ⅓ volume of Protein Precipitation Solution (Gentra Systems) was added and centrifugation was repeated, with the supernatant containing the DNA decanted into a clean tube containing 1 volume of isoproponol. The precipitated DNA was pelleted by centrifugation, rinsed with 1 volume of 70% ethanol, and centrifuged again. The ethanol was removed and the DNA pellet allowed to air dry. The pellet was then resuspended in 50 mM Tris HCl and 10 mM EDTA (pH 8.0). The concentration of the DNA was determined by absorbance at 260 nm. Diluted solutions at 20 ng per μl were prepared for each DNA for use in subsequent PCR reactions.

For certain subjects DNA was extracted from previously established lymphoblastoid cell lines. DNA extraction for these samples also used the GenePure system (Gentra Systems) as described above, except that the red blood cell lysis step was eliminated.

### EXAMPLE 3

Amplification of Polymorphic DNA Markers

PCR amplification and analysis of polymorphic simple sequence repeats (microsatellites) from genomic DNA prepared according to Example 2 was carried out using a modification of the method of Weber and May, Am. J. Hum. Genet. 44:388-396 (1989). Oligonucleotide primers were purchased from Research Gentics or IDT.

PCR was carried out using a MJ Research thermocycler. Each 12 μl reaction contained 40 ng of genomic DNA template, 0.12 units of AmpliTaq Gold polymerase (Perkin Elmer), 12 pmol of each primer, 10 mM Tris-HCl, pH 8.3, 50 mM KCl, 1.5 mM MgCl₂, 0.001% gelatin, 200 mM each of dATP, dGTP, and dTTP, 1.25 μM dCTP, 25 nM ³²p-α-dCTP at 300 Ci/mmole. PCR amplification consisted of an initial denaturation step of 95° for 10 minutes, followed by 25 to 35 cycles of 1 minute denaturation at 94° C., 1 minute annealing at 55° C. to 65° C., and 1 minute extension at 72° C. A final extension at 72° C. for ten minutes was also included. An aliquot of each PCR reaction was mixed with 0.25 volumes of non-denaturing loading buffer and electrophoresed for 2 to 4 hours at 50 W in a 6% nondenaturing polyacrylamide gel. Gels were dried under vacuum and exposed to Kodak X-OMat AR film from 16 to 48 hours. Allele sizes were determined by comparison with PCR products from known genomic DNA standards.

B426K24T is a polymorphic marker developed from the T7 end of BAC 426-K-24 from the California Institute of Technology BAC library, distributed by Research Genetics. BAC end-sequencing was conducted using the Sanger method of dideoxynucleotide termination. Oligonucleotides were designed to amplify a portion of this sequence. The primer sequence of the F primer is 5′-TTTTCTGAGTTCTGTGAATCCTCCTAGTAA-3′, and the R primer sequence is 5′-AATTGATAAAACAACCCATTTAACCAATC-3′. Amplification follows the general protocol above, with the specific annealing temperature of 64° and amplification for 40 cycles.

For analysis of polymorphic markers FcGR2A, FcER1G, and B426K24T, DNA was amplified as above. The PCR product was then mixed with 1 volume of 95% formamide denaturing loading buffer, denatured, snap-cooled on ice, and loaded onto a 0.5× MDE (FMC BioProducts) non-denaturing gel, and electrophoresed at 4 W for 14 hours at room temperature. Gels were dried under vacuum and exposed to Kodak X-OMat AR film from 16 to 48 hours.

### EXAMPLE 4

Linkage Analysis

The cosegregation of polymorphic markers with the schizophrenia phenotype was analyzed for the 22 families noted in Example 1. Standard parametric likelihood analysis was performed by means of FASTLINK [R. W. Cottingham Jr., R. M. Idury, A. A. Schaffer, Am. J. Hum. Genet. 53, 252 (1993)] for two-point linkage and VITESSE [J. R. O'Connel and D. E. Weeks, Nature Genet. 11, 402 (1995)] for multipoint linkage analysis. Multipoint analysis has the advantage of utilizing data from multiple linked markers to maximize the information in a given pedigree, and may also provide better localization of the linked locus. The admixture test as implemented in HOMOG [J. Ott, Analysis of Human Genetic Linkage (Johns Hopkins Univ. Press, Baltimore, 1985), pp. 200-203] was used to test for genetic heterogeneity. To minimize inaccuracies due to errors in pedigree structure, including undetected non-paternity, branches of extended pedigrees that were connected through more than one individual without available DNA were removed from the main pedigrees and analyzed as separate pedigrees. This resulted in 3 small branches (total of 23 individuals) being removed from 3 pedigrees. After this pruning, 89 individuals with no diagnostic or genotype information were needed to accurately represent the pedigree structures of the entire dataset.

Parametric linkage analyses were conducted as they are more powerful than non-parametric methods (Durner et al. Am. J. Hum. Genet. 64:281, 1999) and are robust methods for detecting linkage despite errors or simplifications in the analyzing model, as long as both a dominant and a recessive model are used (Durner et al. 1999, supra; Vieland et al. Hum. Hered. 43:239, 1993; Greenberg et al. Am. J. Hum. Genet. 63:870, 1998). Parametric linkage analysis requires specification of the mode of inheritance. The dominant model was schizophrenia susceptibility allele frequency (p_{A})=0.0045, penetrance of disease (f) of 0.75, 0.50, and 0.001 for disease homozygotes (AA), heterozygotes (Aa), and normal homozygotes (aa), respectively; the recessive model was p_{A}=0.065, f(AA)=0.50, f(Aa)=0.0015, and f(aa)=0.0015. Marker allele frequencies were estimated using a set of 30 unrelated subjects from these families.

In two-point analysis, linkage is assessed between the disease gene and a single marker at a time. Table 4 shows the data from this analysis for the same families and same markers as described above. The results are presented under the hypothesis of heterogeneity. This allows for the possibility that the SCZ gene may not be active in causing illness in every family. The results report the maximum lod score, the recombination fraction (θ) from the markers where the maximum was found, and what proportion of families are estimated as being linked to the SCZ gene (α).

TABLE IV

| | Max Het Lod | | | Max Net Lod | | |
|---|---|---|---|---|---|---|
| Marker | Recessive | θ | α | Dominant | θ | α |
| D1S1653 | 3.52 | 0.1 | 1 | 0.23 | 0.1 | 0.3 |
| D1S398 | 2.10 | 0.1 | 0.9 | 1.23 | 0.05 | 0.65 |
| D1S2705 | 4.26 | 0.1 | 1 | 1.36 | 0.2 | 1 |
| APOA2 | 1.53 | 0.05 | 0.6 | 0.14 | 0 | 0.2 |
| D1S2675 | 1.91 | 0.1 | 0.85 | 1.59 | 0 | 0.6 |
| D1S1679 | 5.79 | 0.05 | 0.95 | 1.57 | 0 | 0.4 |
| D1S2768 | 1.00 | 0.1 | 0.8 | 1.00 | 0 | 0.55 |
| D1S1677 | 2.26 | 0.1 | 0.8 | 0.54 | 0 | 0.3 |

The data indicate a high probability that markers D1S1653, D1S2705, and D1S1679 are linked to SCZ and that the SCZ gene is active in most families. These data also indicate that this locus influences schizophrenia susceptibility in an autosomal recessive fashion, as the lod scores under the recessive model are much higher that those under the dominant model.

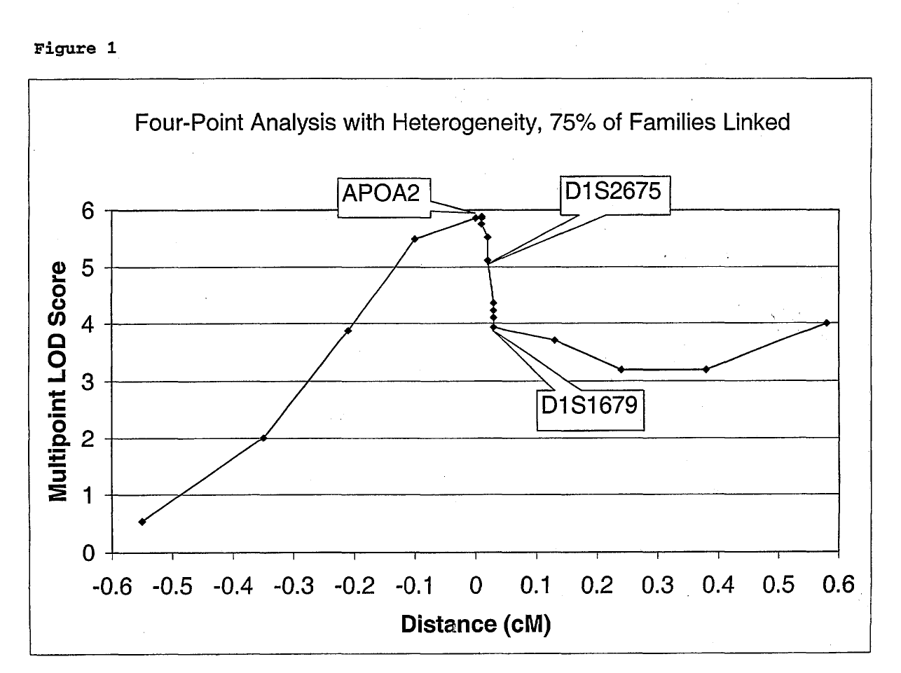

Multipoint linkage analysis considers the genetic data from several markers simultaneously and can provide stronger evidence for linkage and-a better estimate of the position of the gene. FIG. 1 plots the lod score using the markers APOA2, D1S2675, and D1S1679. The maximum lod score under heterogeneity is 5.88, with 75% of families linked. This maximum score, which represents the most likely location of the susceptibility gene, occurs between the markers APOA2 and D1S2675.

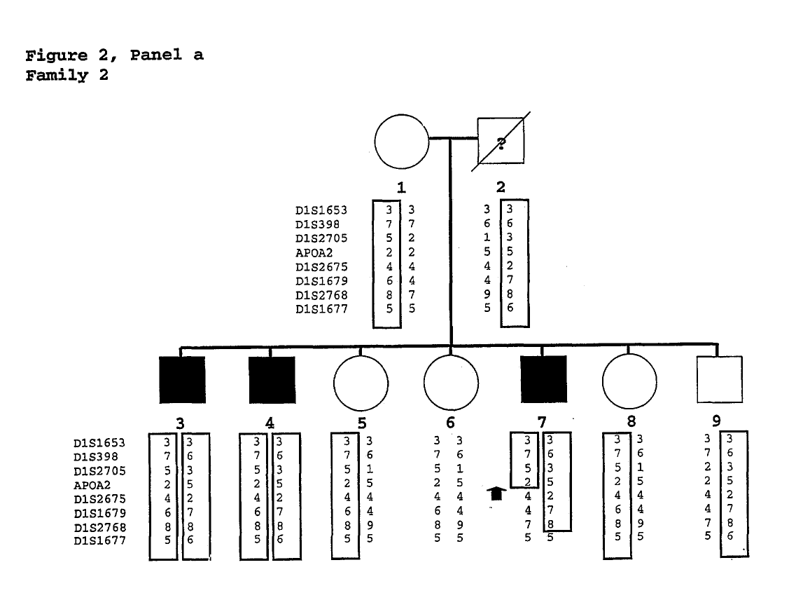

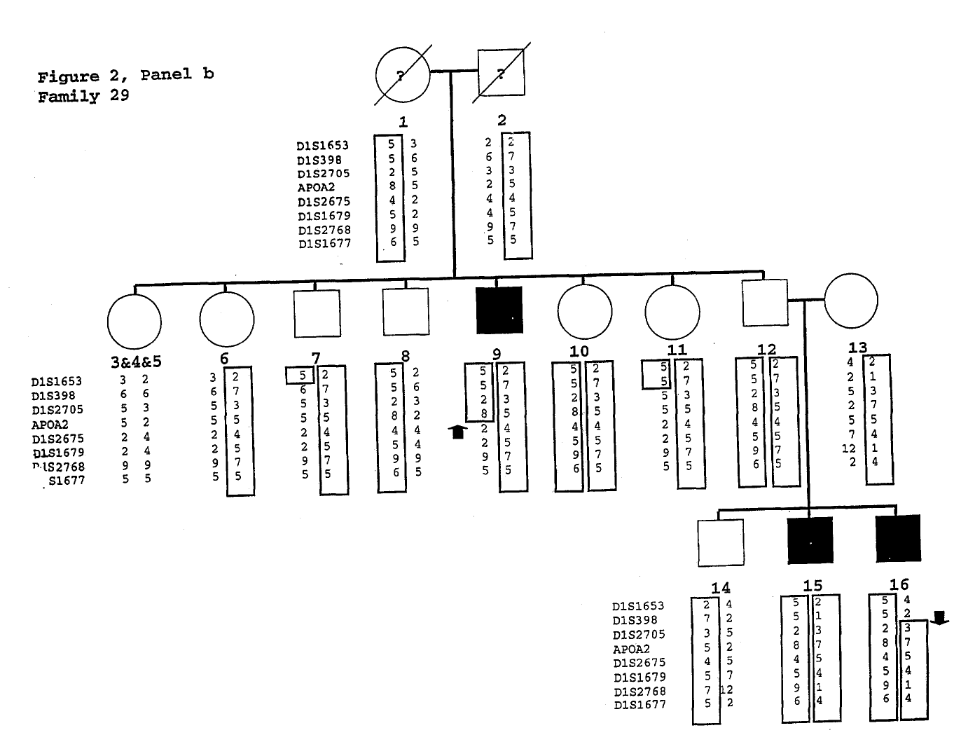

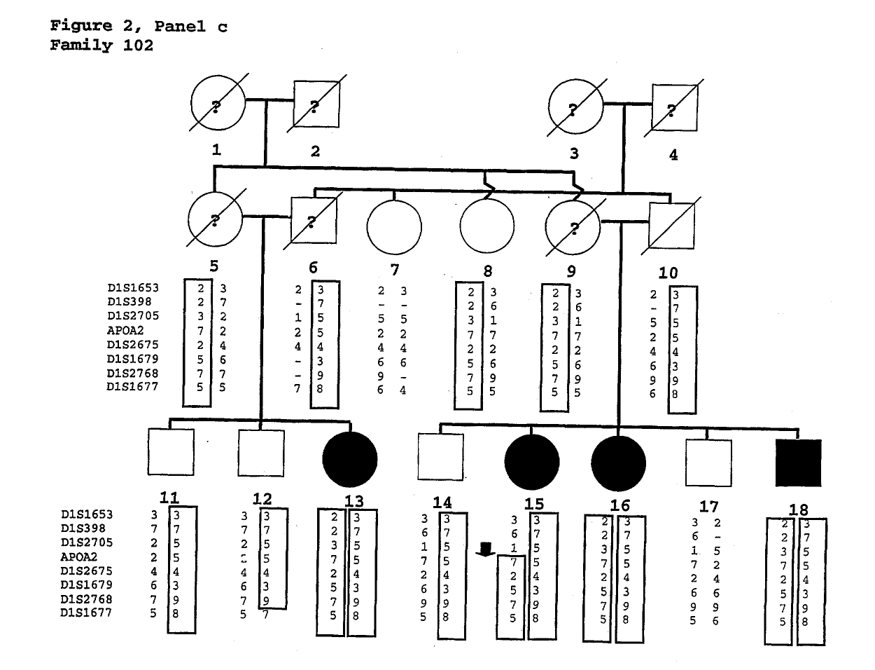

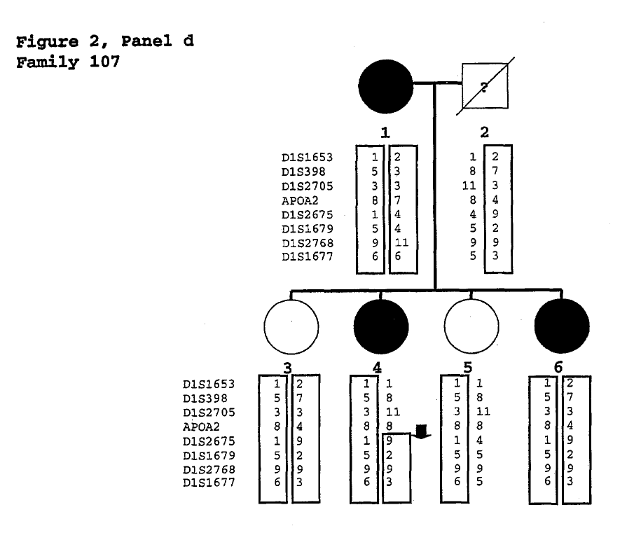

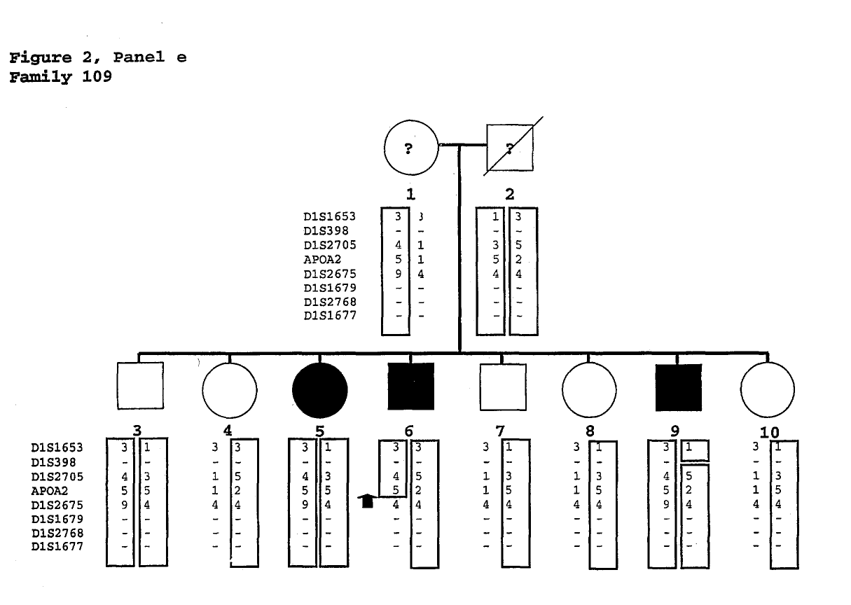

The data have also been subjected to haplotype analysis. This analysis assigns allelic markers between the chromosomes of an individual such that the number of recombination events needed to account for segregation between generations is minimized. In FIG. 2 (panels A-E) illustrating haplotype analysis, boxes represent males and circles represent females. Solid boxes or circles indicate patients or family members who suffer from schizophrenia. Individuals unavailable for diagnosis are marked with a question mark. “1,” 1” 4,” and “9,” for example, represent different allelic variants of the D1S2675 marker. Therefore, for example, in Family 107, the two daughters 107-3 and 107-6 share the complete set of markers from the proposed variant chromosomes of the parents. The daughter 107-5 has inherited only one variant chromosome, and so would be predicted to be a carrier but not express schizophrenia. Daughter 107-4 has two variant chromosomes at the markers D1S2675, D1S1679, D1S2768, and D1S1677. She has only one variant chromosome at markers D1S1653, D1S398, D1S2705, and APOA2. Since 107-4 expresses schizophrenia, and it requires two variant chromosomes from this region of chromosome 1 to be at risk for illness, it is deduced-that SCZ is distal to APOA2. Analysis with additional polymorphic markers in the interval between APOA2 and D1S2675 reveal that SCZ must be distal to the marker B426K24T. Daughter 107-3, while inheriting two variant chromosomes, does not express the illness, and so is nonpenetrant. The genetic model of inheritance predicts that approximately half of individuals inheriting two variant chromosomes will be nonpenetrant or not develop the illness. They are at equal risk as their schiozophrenia siblings of having children with schizophrenia if they marry an individual with at least one variant chromosome 1. Families 002, 029, 102, 107, and 109 all contain recombination events between D1S2705 and D1S1679 that help localize the SCZ gene.

These data indicate that a gene associated with the phenotype of schizophrenia is linked to markers within the chromosome 1q22 region. The pattern of segregation of the disease within the families also serves to confirm the mode of inheritance of the SCZ susceptibility locus is autosomal recessive.

### EXAMPLE 5

BAC Contig Construction

BACs mapping the interval between D1S2705 and D1S1679 are identified by PCR screening of DNA from BAC libraries. DNA pools from the CITB libraries, obtained from Research Genetics, are screened using primers designed from sequence from this interval. Once an individual BAC is identified, it is grown in liquid culture and DNA is extracted. The DNA is used for 1) PCR with all primers in the region to verify identity and overlap, 2) DNA sequencing of both BAC ends, and 3) pulsed-field gel electrophoresis to determine size of the human DNA insert. Once DNA sequence is available, it is used to design new primers that are then used to screen the library again. DNA sequence is also used for homology searches (BLAST) against DNA in the NCBI GENBANK database, which may identify additional overlapping BAC clones. Table 5 lists the BACs known to map to the interval between D1S2705 and D1S1679.

TABLE V
BACs Mapping Between D1S2705 and D1S1679

| BAC Name | Source Library |
|---|---|
| 491-L-16 | CITB |
| 460-O-15 | CITB |
| 2054-K-11 | CITB |
| 426-K-24 | CITB |
| 464-H-15 | RPCI-11 |
| 372-M-11 | CITB |
| 282-K-22 | CITB |
| 3215-C-112 | CITB |
| 2514-J-12 | CITB |
| 3050-G-11 | CITB |
| 3164-M-1 | CITB |
| 465-O-21 | CITB |
| 444-L-5 | CITB |
| 287-G-16 | CITB |
| 316-J-19 | CITB |
| 195-G-14 | RPCI-11 |
| 205-H-1 | RPCI-11 |
| 640-O-16 | RPCI-11 |
| 259-N-10 | CITB |
| 354-J-5 | CITB |
| 2325-L-5 | CITB |
| 978-J-16 | RPCI-11 |
| 921-A-16 | RPCI-11 |
| 456-J-16 | RPCI-11 |
| 990-B-3 | RPCI-11 |
| 3250-P-16 | CITB |
| 130FS | RPCI-11 |
| 565P22 | RPCI-11 |
| 384L19 | RPCI-11 |
| 336H14 | RPCI-11 |
| 227F8 | RPCI-11 |
| 456P18 | RPCI-11 |
| 474716 | RPCI-11 |
| 110D4 | RPCI-11 |
| 25K21 | RPCI-11 |
| 5K23 | RPCI-11 |
| 122G18 | RPCI-11 |
| 297K8 | RPCI-11 |
| 137A12 | RPCI-11 |
| 544M22 | RPCI-11 |
| 381D2 | RPCI-11 |
| 312J18 | RPCI-11 |

The foregoing description of the preferred embodiments of the present invention has been presented for purposes of illustration and description. They are not intended to be exhaustive or to limit the invention to the precise form disclosed, and many modifications and variations are possible in light of the above teaching. All publications and patent applications cited herein are incorporated by reference in their entirety to the same extent as if each individual publication or patent application was specifically and individually so denoted.

What is claimed is:

1. A method of diagnosing susceptibility to schizophrenia in a patient, the method comprising: determining the presence or absence of an allele of a polymorphic marker in the DNA of the patient, wherein the polymorphic marker is within a segment of chromosome 1q22 bordered by D1S2705 and D1S1679 and is linked to a DNA segment (SCZ) having a variant form associated with a phenotype of schizophrenia, and said allele is in phase with the variant form of SCZ, whereby the presence of said allele in the patient indicates susceptibility to schizophrenia.

2. The method of claim 1, wherein the polymorphic marker is APOA2, FcER1G, FcGR2A, B426K24T, or D1S2675.

3. The method of claim 1, wherein the polymorphic marker is within 4 cM of the B426K24T marker.

4. The method of claim 1, wherein the polymorphic marker is between B426K24T and D1S2675.

5. The method of claim 1, wherein the allele is in linkage disequilibrium with the DNA segment.

6. The method of claim 1, further comprising the step of establishing that the allele is in phase with the variant form of the DNA segment.

7. The method of claim 6, wherein the establishing step comprises determining the presence or absence of the allele in first and second degree relatives of the patient, the first and second degree relative each being of known phenotype for schizophrenia, at least one of the relatives having a phenotype of schizophrenia and being informative for the allele.

8. The method of claim 7, further comprising the step of determining the phenotypes of relatives.

9. The method of claim 8, wherein the phenotypes of the relatives are determined by the DSM-IIIR criteria of Table 1 and Table 2.

10. The method of claim 9, wherein one of the relatives is a parent or sibling of the patient.

11. The method of claim 1, further comprising the step of determining the presence or absence of an allele of a second polymorphic marker in the patient.

12. The method of claim 1, wherein the presence or absence of the allele is determined by amplifying a segment of DNA within chromosome 1q22 that spans the polymorphic marker.

13. The method of claim 12, further comprising the step of determining the size of the amplified segment.

14. The method of claim 12, further comprising the step of determining the sequence of the amplified segment.

15. The method of claim 12, further comprising the step of determining the presence or absence of a restriction enzyme site within the amplified segment.

16. The method of claim 1, wherein the presence or absence of the allele is determined by contacting the DNA from the patient with an oligonucleotide probe capable of hybridizing to the allele under stringent conditions; and determining whether hybridization has occurred thereby indicating the presence of the allele.

17. The method of claim 16, further comprising the step of isolating a sample of DNA from the patient.

18. The method of claim 17, wherein the DNA is genomic and the sample is obtained from saliva, blood or buccal mucosal cells.
